(19) United States
(12) Patent Application Publication
(10) Pub. No.: US 2026/0076638 A1
(43) Pub. Date: Mar. 19, 2026

(54) X-Ray Fluoroscopic Imaging Apparatus

(71) Applicant: SHIMADZU CORPORATION, Kyoto-shi (JP)

(72) Inventor: Masahiro TANAKA, Kyoto-shi (JP)

(21) Appl. No.: 19/127,664

(22) PCT Filed: Oct. 13, 2023
(86) PCT No.: PCT/JP2023/037236

§ 371 Date: May 6, 2025

(30) Foreign Application Priority Data
Dec. 22, 2022 (JP) 2022-205684

(51) Int. Cl. A61B 6/00; A61B 6/04; A61B 6/10; A61B 6/46

(52) U.S. Cl. CPC A61B 6/54; A61B 6/0407; A61B 6/107; A61B 6/461; A61B 6/487

(57) ABSTRACT

An X-ray fluoroscopic imaging apparatus (100) includes a table (2), a table operation unit (51), and a controller (31). The controller is configured or programmed to move the table based on an operation input to the table operation unit in a first control performed when movement of the table is not prohibited, and move a center (42a) of an X-ray irradiation field (42) of an X-ray image (41) on a display (40) without moving the table based on the operation input to the table operation unit in a second control performed when the movement of the table is prohibited.

### TECHNICAL FIELD

The present invention relates to an X-ray fluoroscopic imaging apparatus.

### BACKGROUND ART

Conventionally, an X-ray fluoroscopic imaging apparatus including an X-ray irradiator, X-ray detector, and a table is known. Such an X-ray fluoroscopic imaging apparatus is disclosed in Japanese Patent Laid-Open No. 2018-029922, for example.

Japanese Patent Laid-Open No. 2018-029922 discloses an X-ray fluoroscopic imaging system (X-ray fluoroscopic imaging apparatus) including an X-ray tube (X-ray irradiator) that radiates X-rays, an X-ray image detector (X-ray detector) that detects the X-rays radiated to a subject, and a table on which the subject is placed. The X-ray image detector is provided under the table. The X-ray image detector is movable along the longitudinal direction of the table between both ends of the table that extend in the short-side direction of the table.

### PRIOR ART

### Patent Document

Patent Document 1: Japanese Patent Laid-Open No. 2018-029922

### SUMMARY OF THE INVENTION

### Problems to Be Solved by the Invention

In an examination in which an instrument is introduced into the body of a subject, such as an endoscopy, it is preferable for an operator or technician to position a region of interest at the center of an X-ray irradiation field rather than at the periphery from the viewpoint of visibility. However, in such an examination, moving the subject by moving the table while the instrument is introduced into the body of the subject is problematic from the viewpoint of safety, and thus the movement of the table on which the subject is placed may be prohibited during the examination. Thus, even when the position of the region of interest changes depending on the position of the instrument, the center position of the X-ray irradiation field cannot be adjusted by moving the table. Therefore, even when the movement of the table is prohibited during treatment, examination, or the like, it is desired that the center position of the X-ray irradiation field can be adjusted and that the adjustment can be intuitively achieved by the operator or technician.

The present invention is intended to solve at least one of the above problems. The present invention aims to provide an X-ray fluoroscopic imaging apparatus in which the center position of an X-ray irradiation field can be adjusted even when movement of a table is prohibited during treatment, examination, or the like, and the adjustment can be intuitively achieved by an operator or technician.

### Means for Solving the Problems

An X-ray fluoroscopic imaging apparatus according to an aspect of the present invention includes an imager including an X-ray irradiator to irradiate a subject with X-rays and an X-ray detector to detect the X-rays radiated from the X-ray irradiator, an X-ray image generator to generate an X-ray image based on a detection signal detected by the X-ray detector, a table between the X-ray irradiator and the X-ray detector to allow the subject to be placed thereon, the table being movable in a horizontal direction, a table movement mechanism to move the table, a table operation unit to receive an operation input to move the table, a table lock to prohibit movement of the table, a display to display the X-ray image generated by the X-ray image generator, and a controller configured or programmed to control the movement of the table and perform a control to cause the display to display the X-ray image. The controller is configured or programmed to move the table based on the operation input to the table operation unit in a first control performed when the movement of the table is not prohibited by the table lock, and move a center of an X-ray irradiation field of the X-ray image on the display without moving the table based on the operation input to the table operation unit in a second control performed when the movement of the table is prohibited by the table lock.

### Advantageous Effect of the Invention

In the X-ray fluoroscopic imaging apparatus according to this aspect of the present invention, as described above, the controller is configured or programmed to move the table based on the operation input to the table operation unit in the first control performed when the movement of the table is not prohibited by the table lock, and move the center of the X-ray irradiation field of the X-ray image on the display without moving the table based on the operation input to the table operation unit in the second control performed when the movement of the table is prohibited by the table lock. Accordingly, even when the movement of the table is prohibited, the center of the X-ray irradiation field of the X-ray image on the display can be moved by continuing to use the table operation unit that is used to move the table when the movement of the table is not prohibited. Therefore, even when the movement of the table is prohibited during treatment, examination, or the like, the center position of the X-ray irradiation field can be adjusted, and the adjustment can be intuitively achieved by an operator or technician.

### BRIEF DESCRIPTION OF THE DRAWINGS

FIG. 1 is a block diagram showing the configuration of an X-ray fluoroscopic imaging apparatus according to an embodiment.

FIG. 2 is a schematic view showing an X-ray fluoroscopic imaging apparatus body according to the embodiment.

FIG. 3 is a schematic view showing examples of a first group of multiple shielding vanes and a second group of multiple shielding vanes according to the embodiment.

FIG. 4 is a block diagram showing an example of the configurations of a movement mechanism and a rotation mechanism included in the X-ray fluoroscopic imaging apparatus body according to the embodiment.

FIG. 5 is a schematic view showing an example of an operation console according to the embodiment.

FIG. 6 is a schematic explanatory view for illustrating an example of a centering process and a scaling process by a main controller according to the embodiment.

FIG. 7 is a flowchart for illustrating a movement control process and a display control process by the main controller according to the embodiment.

### MODES FOR CARRYING OUT THE INVENTION

An embodiment embodying the present invention is hereinafter described on the basis of the drawings.

### Overall Configuration of X-Ray Fluoroscopic Imaging Apparatus

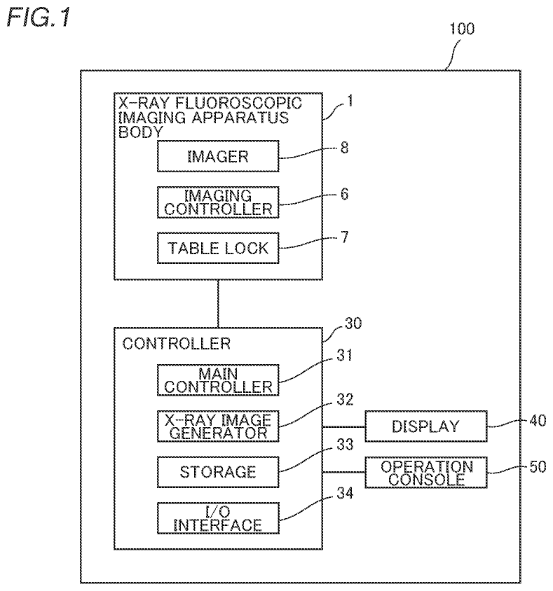

The overall configuration of an X-ray fluoroscopic imaging apparatus 100 according to the embodiment is now described with reference to FIGS. 1 to 5.

As shown in FIG. 1, the X-ray fluoroscopic imaging apparatus 100 is an apparatus that images a subject, which is a human body, using X-rays. The X-ray fluoroscopic imaging apparatus 100 generates an image of the inside of the subject based on the result of imaging of the subject using X-rays.

The X-ray fluoroscopic imaging apparatus 100 includes an X-ray fluoroscopic imaging apparatus body 1 (hereinafter simply referred to as the “apparatus body”), a controller 30, a display 40, and an operation console 50. The apparatus body 1 and the controller 30 are communicably connected to each other. The controller 30 is connected to the display 40 and the operation console 50.

### X-Ray Fluoroscopic Imaging Apparatus Body

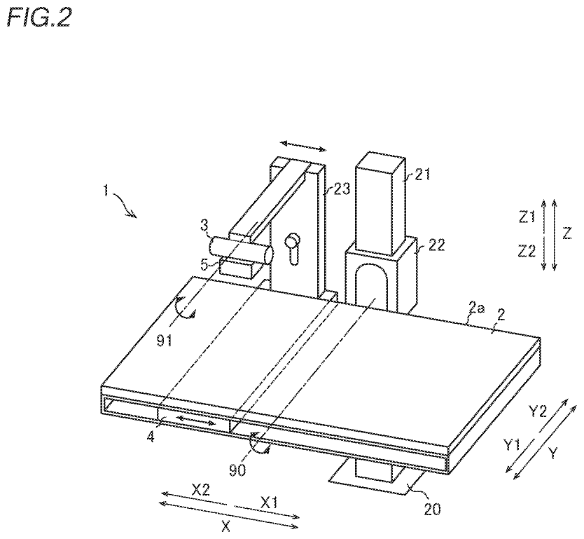

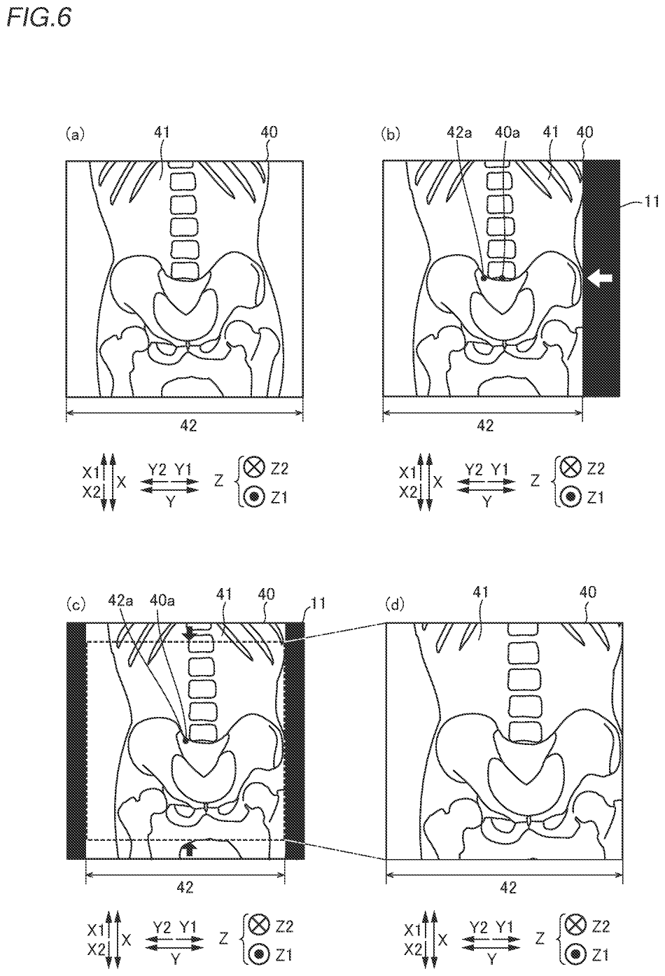

As shown in FIG. 2, the apparatus body 1 includes, as an imaging mechanism, a table 2, an X-ray irradiator 3, an X-ray detector 4, a collimator 5, an imaging controller 6 (see FIG. 1), and a table lock 7 (see FIG. 1). The X-ray irradiator 3 and the X-ray detector 4 constitute an imager 8 that captures an X-ray image 41 (see FIG. 6).

The table 2 includes a surface 2a on which the subject to be imaged (tested) is placed. The table 2 has a substantially rectangular shape as viewed in a direction (Z direction in FIG. 2) substantially perpendicular to the surface 2a. In FIG. 2, the table 2 has a substantially rectangular shape with its long side in an X direction and its short side in a Y direction.

The X-ray irradiator 3 generates X-rays when a high voltage is applied thereto. The X-ray irradiator 3 irradiates the X-ray detector 4 with the generated X-rays. The X-ray irradiator 3 includes an X-ray tube that generates X-rays.

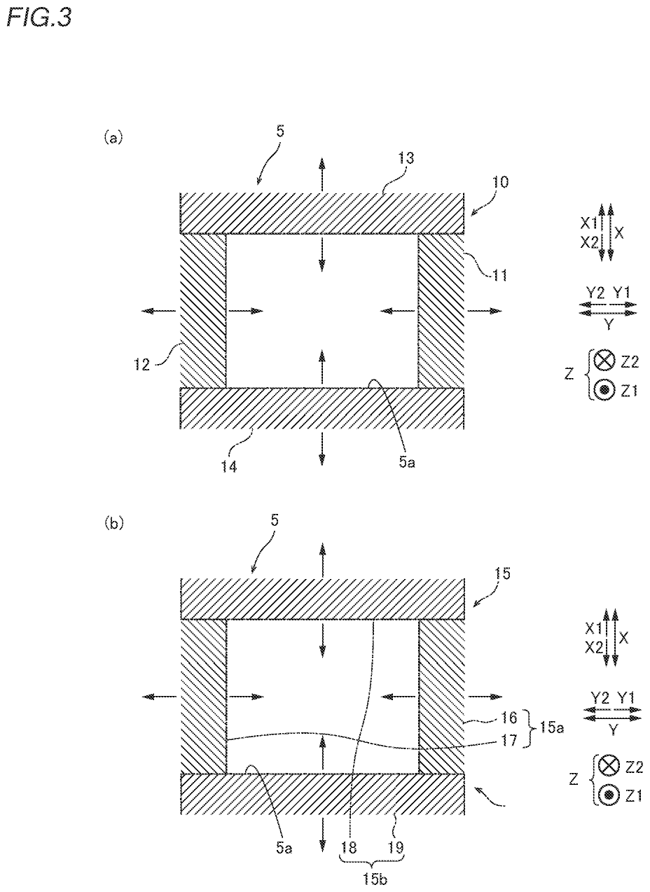

The collimator 5 adjusts an irradiation field 42 (see FIG. 6) of the X-rays generated by the X-ray tube. The collimator 5 is disposed forward in the emission direction of the X-rays. Inside the collimator 5, a first group of multiple shielding vanes 10 provided on the X-ray tube side as shown in FIG. 3(a) and a second group of multiple shielding vanes 15 provided on the table 2 side as shown in FIG. 3(b) are provided. The X-rays radiated from the X-ray tube pass through an opening 5a defined by the first group of multiple shielding vanes 10 and the second group of multiple shielding vanes 15. The collimator 5 is an example of an “irradiation field adjuster” in the claims.

As shown in FIG. 3(a), the first group of multiple shielding vanes 10 includes a first shielding vane 11, a second shielding vane 12, a third shielding vane 13, and a fourth shielding vane 14. The first shielding vane 11 is provided on the Y1 side inside the collimator 5 and is configured to narrow the radiated X-rays by moving in a Y2 direction. The second shielding vane 12 is provided on the Y2 side inside the collimator 5 and is configured to narrow the irradiated X-rays by moving in a Y1 direction. The third shielding vane 13 is provided on the X1 side inside the collimator 5 and is configured to narrow the irradiated X-rays by moving in an X2 direction. The fourth shielding vane 14 is provided on the X2 side inside the collimator 5 and is configured to narrow the irradiated X-rays by moving in an X1 direction.

The first shielding vane 11, the second shielding vane 12, the third shielding vane 13, and the fourth shielding vane 14 are movable independently of each other. Each of the first shielding vane 11, the second shielding vane 12, the third shielding vane 13, and the fourth shielding vane 14 is movable in response to an operation input to a first shielding vane operation unit 52 (see FIG. 5) provided on the operation console 50. Furthermore, the first shielding vane 11 and the second shielding vane 12 are movable based on an operation input to a table operation unit 51 (see FIG. 5) under a second control by a main controller 31 (see FIG. 1) when movement of the table 2 is prohibited by the table lock 7. The second control by the main controller 31 is described below.

As shown in FIG. 3(b), the second group of multiple shielding vanes 15 includes a fifth shielding vane 16, a sixth shielding vane 17, a seventh shielding vane 18, and an eighth shielding vane 19. The fifth shielding vane 16 is provided on the Y1 side inside the collimator 5 and is configured to be able to narrow the irradiated X-rays by moving in the Y2 direction. The sixth shielding vane 17 is provided on the Y2 side inside the collimator 5 and is configured to narrow the irradiated X-rays by moving in the Y1 direction. The fifth shielding vane 16 and the sixth shielding vane 17 are configured as a first pair of shielding vanes 15a that are symmetrically controlled to approach each other or move away from each other along the Y direction.

The seventh shielding vane 18 is provided on the X1 side inside the collimator 5 and is configured to narrow the irradiated X-rays by moving in the X2 direction. The eighth shielding vane 19 is provided on the X2 side inside the collimator 5 and is configured to narrow the irradiated X-rays by moving in the X1 direction. The seventh shielding vane 18 and the eighth shielding vane 19 are configured as a second pair of shielding vanes 15b that are symmetrically controlled to approach each other or move away from each other along the X direction.

The first pair of shielding vanes 15a including the fifth shielding vane 16 and the sixth shielding vane 17, and the second pair of shielding vanes 15b including the seventh shielding vane 18 and the eighth shielding vane 19 are each movable in response to an operation input to a second shielding vane operation unit 53 (see FIG. 5) provided on the operation console 50.

That is, the first group of multiple shielding vanes 10 individually adjusts the position of each side of the irradiation field 42. The second group of multiple shielding vanes 15 adjusts the width (right-left dimension) and length (upward-downward dimension) of the irradiation field 42 without changing the center position of the irradiation field 42.

As shown in FIG. 2, the X-ray detector 4 detects X-rays radiated from the X-ray irradiator 3 and transmitted through the subject. The X-ray detector 4 includes, for example, a flat panel detector (FPD). The X-ray detector 4 transmits a detection signal, which is an electric signal corresponding to the detected X-rays, to an X-ray image generator 32 (see FIG. 1) described below.

In the apparatus body 1, the X-ray irradiator 3 is provided on the front side of the table 2, and the X-ray detector 4 is provided on the rear side of the table 2. The X-ray irradiator 3 and the X-ray detector 4 face each other with the table 2 interposed therebetween.

The imaging controller 6 (see FIG. 1) controls irradiation of X-rays from the apparatus body 1. The imaging controller 6 includes a control device for the X-ray irradiator 3, etc. The imaging controller 6 starts or stops imaging based on an instruction from the main controller 31.

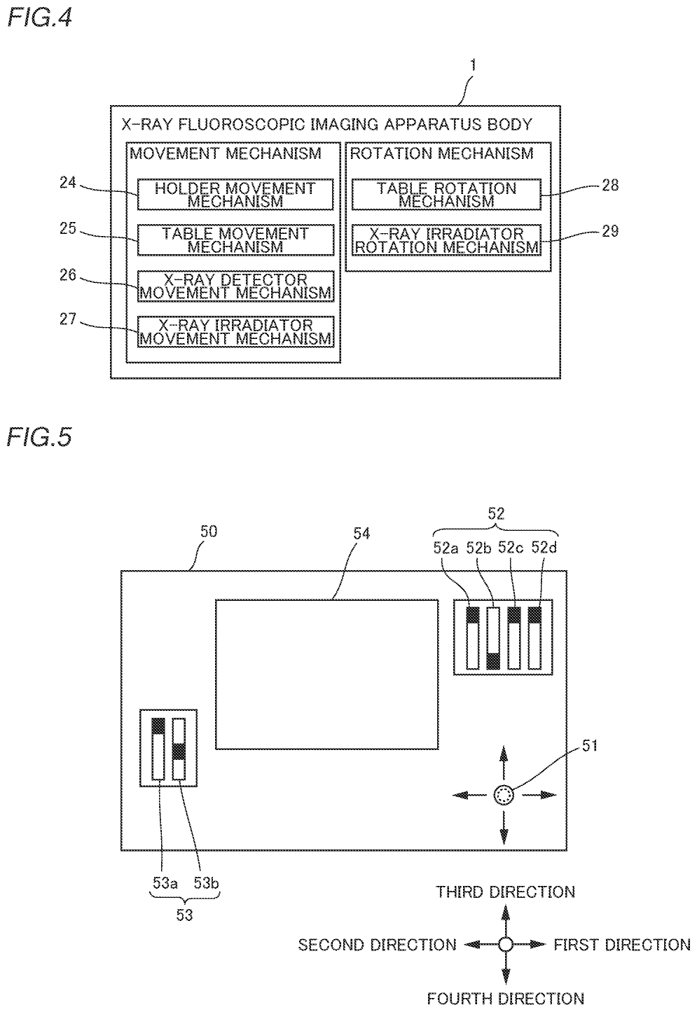

The apparatus body 1 includes, as a support mechanism, a base 20, a first supporting column 21, a holder 22, and a second supporting column 23. As shown in FIG. 4, the apparatus body 1 includes, as a movement mechanism, a holder movement mechanism 24, a table movement mechanism 25, an X-ray detector movement mechanism 26, and an X-ray irradiator movement mechanism 27. The apparatus body 1 also includes, as a rotation mechanism, a table rotation mechanism 28 and an X-ray irradiator rotation mechanism 29.

As shown in FIG. 2, the first supporting column 21 supports the entire apparatus body 1. The first Supporting column 21 is provided on the base 20. The holder movement mechanism 24 (see FIG. 4) is provided on the first supporting column 21. The holder movement mechanism 24 is configured to move the holder 22 in the Z direction.

The holder 22 holds the table 2, the second supporting column 23, and the X-ray detector 4. The table movement mechanism 25 (see FIG. 4), the X-ray detector movement mechanism 26 (see FIG. 4), and the table rotation mechanism 28 (see FIG. 4) are provided on the holder 22. The table movement mechanism 25 is configured to move the table 2 in the short-side direction (Y direction in FIG. 2) of the table 2. The X-ray detector movement mechanism 26 is configured to move the X-ray detector 4 in the longitudinal direction (X direction in FIG. 2) of the table 2. An X-Y direction in FIG. 2 is a substantially horizontal direction. The table rotation mechanism 28 is configured to rotate the table 2 around an axis 90 extending along the short-side direction (Y direction) of the table 2.

The second supporting column 23 supports the X-ray irradiator 3. The X-ray irradiator movement mechanism 27 (see FIG. 4) and the X-ray irradiator rotation mechanism 29 (see FIG. 4) are provided on the second supporting column 23. The X-ray irradiator movement mechanism 27 is configured to move the X-ray irradiator 3 in the longitudinal direction (X direction in FIG. 2) of the table 2. The imager 8 including the X-ray irradiator 3 and the X-ray detector 4 can move integrally with respect to the table 2 by the synchronous operation of the X-ray irradiator movement mechanism 27 and the X-ray detector movement mechanism 26. The X-ray irradiator rotation mechanism 29 is configured to rotate the X-ray irradiator 3 around an axis 91 extending along the short-side direction (Y direction) of the table 2.

The table lock 7 (see FIG. 1) is configured to lock the table 2 such that the table 2 cannot be moved with respect to the holder 22. The table lock 7 can switch between a locked state in which the table 2 cannot be moved with respect to the holder 22 and an unlocked state in which the table 2 can be moved with respect to the holder 22. The table lock 7 is configured to, for example, cancel the magnetic force of a permanent magnet by energizing an electromagnet using the magnetic force of the permanent magnet to weaken a restriction on movement of the table 2 with respect to the holder 22. In other words, when the electromagnet is not energized, movement of the table 2 in the short-side direction (Y direction in FIG. 2) with respect to the holder 22 is restricted (locked). The table lock 7 can use a known configuration capable of restricting movement of the table 2 in the short-side direction with respect to the holder 22, and the configuration of the table lock 7 is not particularly limited.

### Controller, Display, and Operation Console

As shown in FIG. 1, the controller 30 includes a personal computer (PC), for example. The controller 30 includes the main controller 31, the X-ray image generator 32, a storage 33, and an I/O interface 34. The controller 30 is connected to the display 40 and the operation console 50. The main controller 31 is an example of a “controller” in the claims.

The main controller 31 includes a processor such as a central processing unit (CPU), and controls the operation of the X-ray fluoroscopic imaging apparatus 100, including controlling movement of the table 2 and controlling display of the X-ray image 41 on the display 40, by executing application programs stored in the storage 33.

The main controller 31 performs a first control to move the table 2 (see FIG. 2) based on an operation input to the table operation unit 51 (see FIG. 5) when movement of the table 2 is not prohibited by the table lock 7, and performs a second control to move the center 42a (see FIG. 6) of the irradiation field 42 of the X-ray image 41 on the display 40 without moving the table 2 based on an operation input to the table operation unit 51 when movement of the table 2 is prohibited by the table lock 7.

The X-ray image generator 32 includes a processor such as a graphics processing unit (GPU) or a field-programmable gate array (FPGA) configured for X-ray image generation. The X-ray image generator 32 generates the X-ray image 41 based on a detection signal detected by the X-ray detector 4.

The storage 33 includes a volatile storage device and a non-volatile storage device, and stores application programs, etc.

The I/O interface 34 includes various interfaces to input and output signals to and from the controller 30. The I/O interface 34 is connected to the display 40 and the operation console 50.

The display 40 is, for example, a liquid crystal display. The display 40 is configured to display the X-ray image 41 generated by the X-ray image generator 32, etc.

As shown in FIG. 5, the operation console 50 includes the table operation unit 51, the first shielding vane operation unit 52, the second shielding vane operation unit 53, and an input 54.

The table operation unit 51 includes a joystick, for example. The table operation unit 51 is configured to be tilted in a first direction (right direction in FIG. 5), a second direction (left direction in FIG. 5), a third direction (upward direction in FIG. 5), and a fourth direction (downward direction in FIG. 5) such that a direction corresponding to the tilted direction can be input. The first control to move the table 2 and the second control to move the center 42a (see FIG. 6) of the irradiation field 42 of the X-ray image 41 based on the operation input to the table operation unit 51 are described below in detail.

The first shielding vane operation unit 52 receives an operation input to move each of the first shielding vane 11, the second shielding vane 12, the third shielding vane 13, and the fourth shielding vane 14. The first shielding vane operation unit 52 includes, for example, four knobs that correspond to the first shielding vane 11, the second shielding vane 12, the third shielding vane 13, and the fourth shielding vane 14, respectively, and are movable in a forward-rearward direction (upward-downward direction in FIG. 5).

The first shielding vane operation unit 52 includes a first knob 52a, a second knob 52b, a third knob 52c, and a fourth knob 52d. The first knob 52a receives an operation input to move the first shielding vane 11 in the Y2 direction to narrow one side of the X-ray irradiation field 42 in the Y1 direction, and receives an operation input to move the first shielding vane 11 in the Y1 direction to release the narrowing. The second knob 52b receives an operation input to move the second shielding vane 12 in the Y1 direction to narrow one side of the X-ray irradiation field 42 in the Y2 direction, and receives an operation input to move the second shielding vane 12 in the Y2 direction to release the narrowing. The third knob 52c receives an operation input to move the third shielding vane 13 in the X2 direction to narrow one side of the X-ray irradiation field 42 in the X1 direction, and receives an operation input to move the third shielding vane 13 in the X1 direction to release the narrowing. The fourth knob 52d receives an operation input to move the fourth shielding vane 14 in the X1 direction to narrow one side of the X-ray irradiation field 42 in the X2 direction, and receives an operation input to move the fourth shielding vane 14 in the X2 direction to release the narrowing.

The second shielding vane operation unit 53 receives operation inputs to move the first pair of shielding vanes 15a including the fifth shielding vane 16 and the sixth shielding vane 17, and the second pair of shielding vanes 15b including the seventh shielding vane 18 and the eighth shielding vane 19. The second shielding vane operation unit 53 includes, for example, two knobs that correspond to the first pair of shielding vanes 15a and the second pair of shielding vanes 15b, respectively, and are movable in the forward-rearward direction (upward-downward direction in FIG. 5).

The second shielding vane operation unit 53 includes a fifth knob 53a and a sixth knob 53b. The fifth knob 53a receives an operation input to move the first pair of shielding vanes 15a toward each other along the Y direction to narrow two sides of the X-ray irradiation field 42 in the Y direction, and receives an operation input to move the first pair of shielding vanes 15a away from each other along the Y direction to release the narrowing. The sixth knob 53b receives an operation input to move the second pair of shielding vanes 15b toward each other along the X direction to narrow two sides of the X-ray irradiation field 42 in the X direction, and receives an operation input to move the second pair of shielding vanes 15b away from each other along the X direction to release the narrowing.

The input 54 is, for example, a touch panel. The input 54 receives an input operation by an operator or technician to operate the X-ray fluoroscopic imaging apparatus 100. For example, the input 54 receives operation inputs to cause the table lock 7 to lock and unlock movement of the table 2 in the Y direction.

### First Control and Second Control

The main controller 31 (see FIG. 1) performs the first control when movement of the table 2 is not prohibited by the table lock 7. The main controller 31 performs the first control based on the input 54 receiving an operation input to unlock the table 2, for example. In the first control, the main controller 31 moves the table 2 or the imager 8 including the X-ray irradiator 3 and the X-ray detector 4 based on an operation input to the table operation unit 51.

In the first control, the main controller 31 (see FIG. 1) associates the first direction of the table operation unit 51 with the Y1 direction (see FIG. 2) as the moving direction of the table 2. In the first control, the main controller 31 moves the table 2 in the Y1 direction when the table operation unit 51 is tilted in the first direction. At this time, when the X-ray image 41 is displayed on the display 40, the X-ray image 41 is scrolled in the Y1 direction on the display 40. In addition, in the first control, the main controller 31 associates the second direction of the table operation unit 51 with the Y2 direction (see FIG. 2) as the moving direction of the table 2. In the first control, the main controller 31 moves the table 2 in the Y2 direction when the table operation unit 51 is tilted in the second direction. At this time, when the X-ray image 41 is displayed on the display 40, the X-ray image 41 is scrolled in the Y2 direction on the display 40.

In the first control, the main controller 31 (see FIG. 1) associates the third direction of the table operation unit 51 with the X1 direction (see FIG. 2) as the moving direction of the imager 8. In the first control, the main controller 31 moves the imager 8 in the X1 direction when the table operation unit 51 is tilted in the third direction. In the first control, the main controller 31 also associates the fourth direction of the table operation unit 51 with the X2 direction (see FIG. 2) as the moving direction of the imager 8. In the first control, the main controller 31 moves the imager 8 in the X2 direction when the table operation unit 51 is tilted in the fourth direction.

The main controller 31 (see FIG. 1) performs the second control when movement of the table 2 is prohibited by the table lock 7. The main controller 31 performs the second control based on the input 54 receiving an operation input to lock the table 2, for example. In the second control, the main controller 31 moves the first shielding vane 11, the second shielding vane 12, or the imager 8 without moving the table 2 based on the operation input to the table operation unit 51.

In the second control, the main controller 31 (see FIG. 1) associates the first direction of the table operation unit 51 with the Y2 direction (see FIG. 3(a)) as the moving direction of the first shielding vane 11. In the second control, the main controller 31 moves the first shielding vane 11 in the Y2 direction when the table operation unit 51 is tilted in the first direction. Thus, the main controller 31 performs a control to move the first shielding vane 11 to adjust the irradiation field 42 to move the center 42a of the irradiation field 42 of the X-ray image 41 on the display 40 in the second direction (Y2 direction) opposite to the first direction indicated by the operation input to the table operation unit 51.

Furthermore, in the second control, the main controller 31 associates the second direction of the table operation unit 51 with the Y1 direction (see FIG. 3(a)) as the moving direction of the first shielding vane 11 when the first shielding vane 11 has already been moved in the Y2 direction. In the second control, the main controller 31 moves the first shielding vane 11 in the Y1 direction when the first shielding vane 11 has already been moved in the Y2 direction and the table operation unit 51 is tilted in the second direction.

In the second control, the main controller 31 (see FIG. 1) associates the second direction of the table operation unit 51 with the Y1 direction (see FIG. 3(a)) as the moving direction of the second shielding vane 12. In the second control, the main controller 31 moves the second shielding vane 12 in the Y1 direction when the table operation unit 51 is tilted in the second direction. Thus, the main controller 31 performs a control to move the second shielding vane 12 to adjust the irradiation field 42 to move the center 42a of the irradiation field 42 of the X-ray image 41 on the display 40 in the first direction (Y1 direction) opposite to the second direction indicated by the operation input to the table operation unit 51. Furthermore, in the second control, the main controller 31 associates the first direction of the table operation unit 51 with the Y2 direction (see FIG. 3(a)) as the moving direction of the second shielding vane 12 when the second shielding vane 12 has already been moved in the Y1 direction. In the second control, the main controller 31 moves the second shielding vane 12 in the Y2 direction when the second shielding vane 12 has been moved in the Y1 direction and the table operation unit 51 is tilted in the first direction.

In the second control, the main controller 31 (see FIG. 1) moves the center 42a of the irradiation field 42 of the X-ray image 41 on the display 40 in a direction opposite to the moving direction indicated by the operation input to the table operation unit 51, in accordance with the amount of movement in the Y direction of the first shielding vane 11 or the second shielding vane 12.

In the second control, the main controller 31 (see FIG. 1) moves only one of the first shielding vane 11 and the second shielding vane 12 when the table operation unit 51 is tilted in one of the first direction and the second direction.

In the second control, the main controller 31 (see FIG. 1) associates the third direction of the table operation unit 51 with the X1 direction (see FIG. 2) as the moving direction of the imager 8, similarly to the first control. In the first control, the main controller 31 moves the imager 8 in the X1 direction when the table operation unit 51 is tilted in the third direction. In the second control, the main controller 31 also associates the fourth direction of the table operation unit 51 with the X2 direction (see FIG. 2) as the moving direction of the imager 8, similarly to the first control. In the first control, the main controller 31 moves the imager 8 in the X2 direction when the table operation unit 51 is tilted in the fourth direction.

### Centering Process of X-Ray Image in Second Control

In the second control, the main controller 31 (see FIG. 1) performs a centering process to align the center 42a of the irradiation field 42 of the X-ray image 41 with the center 40a of the display 40 by moving the X-ray image 41 in the Y direction, as shown in FIG. 6(c), when moving the center 42a of the irradiation field 42 of the X-ray image 41 on the display 40. The irradiation field 42 refers to a range of X-ray irradiation that is not blocked by the shielding vanes.

In the second control, as shown in FIG. 6(b), the main controller 31 (see FIG. 1) moves the first shielding vane 11 in the Y2 direction when the table operation unit 51 is tilted in the first direction. Thus, the irradiation field 42 narrowed by the first shielding vane 11 is displayed with a bias in the Y2 direction on the display 40. As shown in FIG. 6(c), the main controller 31 displays the irradiation field 42 displayed with a bias in the Y2 direction on the display 40 at the approximate center of the display 40 in the Y direction by performing a control to move the X-ray image 41 in the Y1 direction to align the center 42a of the irradiation field 42 of the X-ray image 41 with the center 40a of the display 40. The black area at the left end of the display 40 in FIG. 6(c) is an area in which the X-ray image 41 is not displayed.

In the second control, the main controller 31 (see FIG. 1) displays the irradiation field 42 displayed with a bias in the Y1 direction on the display 40 at the approximate center of the display 40 in the Y direction when the table operation unit 51 is tilted in the second direction.

X-Ray Image Scaling Process in Second Control In the second control, as shown in FIG. 6(d), the main controller 31 (see FIG. 1) performs a scaling process to scale up and down an image of the adjusted irradiation field 42 while maintaining the aspect ratio of the X-ray image 41.

When the operator or technician operates the sixth knob 53b of the second shielding vane operation unit 53 to move the second pair of shielding vanes 15b toward each other in the X direction as shown by the arrows in FIG. 6(c) to narrow the two X-direction sides of the irradiation field 42, the main controller 31 increases the X-direction and Y-direction sizes of the adjusted irradiation field 42 while maintaining the X-direction and Y-direction ratio of the X-ray image 41 such that the X-direction size of the irradiation field 42 with the two narrowed X-direction sides matches the vertical screen size of the display 40, as shown in FIG. 6(d).

That is, the main controller 31 increases the X-direction and Y-direction sizes of the irradiation field 42 while maintaining the X-direction and Y-direction ratio of the X-ray image 41 so as to align the two narrowed X-direction sides of the irradiation field 42 with the two vertical sides of the display 40. The “screen size of the display 40” refers to the size of the display area of the X-ray image 41 on the display 40, including the full-screen display.

When the X-direction and Y-direction ratio of the narrowed irradiation field 42 does not match the aspect ratio of the screen size, the settings may be such that priority is given to matching the X-direction size of the irradiation field 42 with the vertical screen size of the display 40, or that priority is given to matching the Y-direction size of the irradiation field 42 with the horizontal screen size of the display 40.

For example, in the second control, the main controller 31 (see FIG. 1) reduces the X-direction and Y-direction sizes of the adjusted irradiation field 42 while maintaining the X-direction and Y-direction ratio of the X-ray image 41 such that the X-direction size of the unnarrowed irradiation field 42 matches the vertical screen size of the display 40, when the first shielding vane 11 has already been moved in the Y2 direction and the table operation unit 51 is tilted in the second direction, and when the operator or technician operates the sixth knob 53b of the second shielding vane operation unit 53 to move the second pair of shielding vanes 15b away from each other to release the narrowing. That is, the main controller 31 reduces the X-direction and Y-direction sizes of the irradiation field 42 while maintaining the X-direction and Y-direction ratio of the X-ray image 41 so as to align the two narrowed X-direction sides of the irradiation field 42 with the two vertical sides of the display 40.

### Operation Input to Table Operation Unit and Display of X-Ray Image in Second Control

An operation input to the table operation unit 51 and display of the X-ray image 41 in the second control are now described with reference to FIG. 6(a) to 6(d). As an example of this embodiment, display of the X-ray image 41 on the display 40 based on an operation input by tilting the table operation unit 51 in the first direction in the second control is described below.

As shown in FIG. 6(a), in a state in which movement of the table 2 is prohibited by the table lock 7, the X-ray image 41 is displayed on the display 40. At this time, the table operation unit 51 is not tilted in any of the first to fourth directions. None of the first to fourth shielding vanes 11 to 14 and the fifth to eighth shielding vanes 16 to 19 are moved to narrow the radiated X-rays. The imaging range of the X-ray image 41 in the initial state displayed on the display 40 shown in FIG. 6(a) is substantially the same as the detection range of the FPD of the X-ray detector 4.

Then, the table operation unit 51 is tilted in the first direction such that an operation input in the first direction is executed on the table operation unit 51. The main controller 31 performs a control to move the first shielding vane 11 in the second direction to adjust the irradiation field 42, based on the operation input in the first direction to the table operation unit 51. Thus, an X-ray shielded region corresponding to the first shielding vane 11 is formed at the right end of the display 40, and the center 42a of the irradiation field 42 of the X-ray image 41 on the display 40 is moved in the second direction. That is, as shown in FIG. 6(b), the irradiation field 42 narrowed by the first shielding vane 11 is displayed with a bias in the Y2 direction on the display 40.

Then, as shown in FIG. 6(c), the main controller 31 performs a control to move the X-ray image 41 in the Y1 direction to align the center 42a of the irradiation field 42 of the X-ray image 41 with the center 40a of the display 40 to display the irradiation field 42 displayed with a bias in the Y2 direction on the display 40 at the approximate center of the display 40 in the Y direction.

Based on the operation input in the first direction to the table operation unit 51, the irradiation field 42 displayed with a bias in the Y2 direction on the display 40 is displayed at the approximate center of the display 40 in the Y direction, and thus the adjusted irradiation field 42 moves in the Y1 direction on the display 40.

In the first control, when an operation in the first direction is input to the table operation unit 51, the table 2 moves in the Y1 direction, and the subject moves to the left in the X-ray image 41 on the display 40. In the second control, when an operation in the first direction is input to the table operation unit 51, the right end of the X-ray image 41 is scraped off, and the entire X-ray image including the subject moves to the left as if the table 2 had moved to the left as in the first control, although the table 2 has not moved. That is, the scroll direction on the display 40 in response to the operation on the table operation unit 51 in the first control coincides with the scroll direction on the display 40 in response to the operation on the table operation unit 51 in the second control. Therefore, based on the operation input in the first direction to the table operation unit 51, the operator or technician can be caused to visually recognize on the display 40 the table 2, which is prohibited from moving, as if it were moving in the Y1 direction.

Then, the two sides of the irradiation field 42 in the X direction are narrowed based on the operation input to the sixth knob 53b of the second shielding vane operation unit 53, and the main controller 31 increases the X-direction and Y-direction sizes of the adjusted irradiation field 42 while maintaining the X-direction and Y-direction ratio of the X-ray image 41 such that the X-direction size of the irradiation field 42 with the two narrowed X-direction sides matches the vertical screen size of the display 40, as shown in FIG. 6(d). With the above, the second control by the main controller 31 based on the operation input in the first direction to the table operation unit 51 is terminated.

It is also possible to revert the display of the X-ray image 41 displayed on the display 40 shown in FIG. 6(d) to the display of the X-ray image 41 displayed on the display 40 shown in FIG. 6(a) by executing the above-described operations and controls in the reverse order. Furthermore, by further executing the above-described operations and controls from the display of the X-ray image 41 displayed on the display 40 shown in FIG. 6(d), it is also possible to further move the center 42a of the irradiation field 42 of the X-ray image 41 displayed on the display 40 shown in FIG. 6(d) in the Y2 direction, then align the center 42a of the irradiation field 42 of the X-ray image 41 with the center 40a of the display 40, and further increase the size of the adjusted irradiation field 42.

In the second control, the display of the X-ray image 41 on the display 40 by tilting the table operation unit 51 in the second direction is similar to the display of the X-ray image 41 on the display 40 by tilting the table operation unit 51 in the first direction described above.

That is, when the table operation unit 51 is not tilted and the first to eighth shielding vanes 11 to 19 are not moved, the table operation unit 51 is tilted in the second direction such that an operation input in the second direction is executed on the table operation unit 51. The main controller 31 performs a control to move the second shielding vane 12 in the first direction to adjust the irradiation field 42, based on the operation input in the second direction to the table operation unit 51. Thus, the center 42a of the irradiation field 42 of the X-ray image 41 on the display 40 is moved in the first direction. The main controller 31 then performs a control to align the center 42a of the irradiation field 42 of the X-ray image 41 with the center 40a of the display 40 to display the irradiation field 42 displayed with a bias in the Y1 direction on the display 40 at the approximate center of the display 40 in the Y direction.

Based on the operation input in the second direction to the table operation unit 51, the irradiation field 42 displayed with a bias in the Y1 direction on the display 40 is displayed at the approximate center of the display 40 in the Y direction, and thus the adjusted irradiation field 42 moves in the Y2 direction on the display 40. Therefore, based on the operation input in the second direction to the table operation unit 51, the operator or technician can be caused to visually recognize on the display 40 the table 2, which is prohibited from moving, as if it were moving in the Y2 direction.

Movement Control Process and Display Control Process by Main Controller

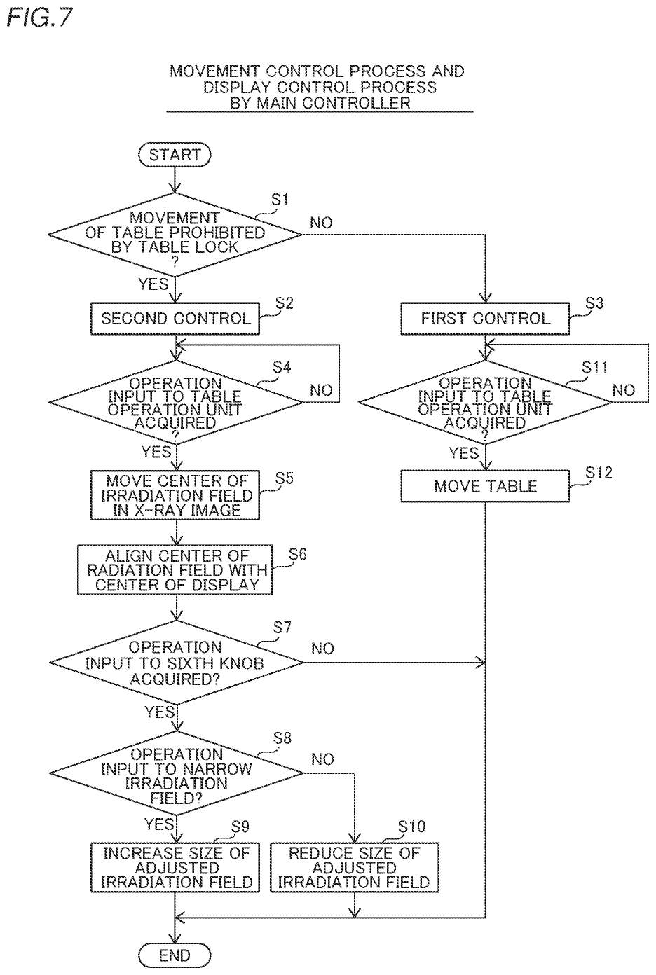

A movement control process and a display control process by the main controller 31 according to this embodiment are now described with reference to FIG. 7. The order of the process steps can be reversed or executed simultaneously as long as there are no contradictions.

In step S1, the main controller 31 determines whether or not movement of the table 2 is prohibited by the table lock 7. When the main controller 31 determines that movement of the table 2 is prohibited by the table lock 7 (Yes in step S1), the process advances to step S2, and when the main controller 31 determines that movement of the table 2 is not prohibited by the table lock 7 (No in step S1), the process advances to step S3.

In step S2, the main controller 31 performs the second control based on the operation input to the table operation unit 51. Specifically, the main controller 31 performs a process to move the center 42a of the irradiation field 42 of the X-ray image 41 on the display 40 without moving the table 2 based on the operation input to the table operation unit 51. Thereafter, the process advances to step S4.

In step S3, the main controller 31 performs the first control based on the operation input to the table operation unit 51. Specifically, the main controller 31 performs a process to move the table 2 based on the operation input to the table operation unit 51. Thereafter, the process is terminated.

In step S4, the main controller 31 determines whether or not an operation input in the first direction or the second direction to the table operation unit 51 has been acquired. When the main controller 31 has acquired an operation input in the first direction or the second direction to the table operation unit 51 (Yes in step S4), the process advances to step S5, and when the main controller 31 determines that an operation input in the first direction or the second direction to the table operation unit 51 has not been acquired (No in step S4), the process advances to step S4.

In step S5, the main controller 31 performs a control to cause the collimator 5 to adjust the irradiation field 42 to move the center 42a of the irradiation field 42 of the X-ray image 41 on the display 40 in the direction opposite to the moving direction indicated by the operation input to the table operation unit 51. Thereafter, the process advances to step S6.

In step S6, the main controller 31 moves the X-ray image 41 in the Y direction to align the center 42a of the irradiation field 42 of the X-ray image 41 with the center 40a of the display 40. Thereafter, the process advances to step S7.

In step S7, the main controller 31 determines whether or not an operation input to the sixth knob 53b for the second pair of shielding vanes 15b has been acquired. When the main controller 31 has acquired an operation input to the sixth knob 53b for the second pair of shielding vanes 15b (Yes in step S7), the process advances to step S8, and when the main controller 31 determines that an operation input to the sixth knob 53b for the second pair of shielding vanes 15b has not been acquired (No in step S7), the process is terminated.

In step S8, the main controller 31 determines whether or not the acquired operation input to the sixth knob 53b is an operation input to move the second pair of shielding vanes 15b toward each other along the X direction to narrow the two X-direction sides of the X-ray irradiation field 42. When the main controller 31 determines that the acquired operation input is an operation input to narrow the two X-direction sides of the X-ray irradiation field 42 (Yes in step S8), the process advances to step S9, and when the main controller 31 determines that the acquired operation input is not an operation input to narrow the two X-direction sides of the X-ray irradiation field 42 but an operation input to move the two X-direction sides of the X-ray irradiation field 42 away from each other to release the narrowing (No in step S8), the process advances to step S10.

In step S9, the main controller 31 increases the size of the adjusted irradiation field 42 such that the X-direction size of the irradiation field 42 with the two narrowed X-direction sides matches the vertical screen size of the display 40. Thereafter, the process is terminated.

In step S10, the main controller 31 reduces the size of the adjusted irradiation field 42 such that the X-direction size of the unnarrowed irradiation field 42 matches the vertical screen size of the display 40. Thereafter, the process is terminated.

In step S11, the main controller 31 determines whether or not an operation input in the first direction or the second direction to the table operation unit 51 has been acquired. When the main controller 31 has acquired an operation input in the first direction or the second direction to the table operation unit 51 (Yes in step S11), the process advances to step S12, and when the main controller 31 determines that an operation input in the first direction or the second direction to the table operation unit 51 has not been acquired (No in step S11), the process advances to step S11.

In step S12, the main controller 31 moves the table 2 in the moving direction indicated by the operation input to the table operation unit 51. Thereafter, the process is terminated.

### Advantages of This Embodiment

In this embodiment, the following advantages are obtained.

In this embodiment, as described above, the X-ray fluoroscopic imaging apparatus 100 includes the imager 8 including the X-ray irradiator 3 to irradiate a subject with X-rays and an X-ray detector 4 to detect the X-rays radiated from the X-ray irradiator 3, the X-ray image generator 32 to generate the X-ray image 41 based on the detection signal detected by the X-ray detector 4, the table 2 between the X-ray irradiator 3 and the X-ray detector 4 to allow the subject to be placed thereon and movable in the horizontal direction, the table movement mechanism 25 to move the table 2, the table operation unit 51 to receive an operation input to move the table 2, the table lock 7 to prohibit movement of the table 2, the display 40 to display the X-ray image 41 generated by the X-ray image generator 32, and the main controller 31 configured or programmed to control movement of the table 2 and perform a control to cause the display 40 to display the X-ray image 41. The main controller 31 is configured or programmed to move the table 2 based on the operation input to the table operation unit 51 in the first control performed when movement of the table 2 is not prohibited by the table lock 7, and move the center 42a of the X-ray irradiation field 42 of the X-ray image 41 on the display 40 without moving the table 2 based on the operation input to the table operation unit 51 in the second control performed when movement of the table 2 is prohibited by the table lock 7. Accordingly, even when movement of the table 2 is prohibited, the center 42a of the X-ray irradiation field 42 of the X-ray image 41 on the display 40 can be moved by continuing to use the table operation unit 51 that is used to move the table 2 when movement of the table 2 is not prohibited. Therefore, even when movement of the table 2 is prohibited during treatment, examination, or the like, the center position of the X-ray irradiation field 42 can be adjusted, and the adjustment can be intuitively achieved by the operator or technician.

In the embodiment described above, with the following configurations, the following advantages are further obtained.

That is, in this embodiment, as described above, the main controller 31 is configured or programmed to move the center 42a of the irradiation field 42 of the X-ray image 41 on the display 40 in the direction opposite to the moving direction indicated by the operation input to the table operation unit 51 in the second control. Accordingly, the moving direction indicated by the operation input to the table operation unit 51 can coincide with the moving direction of the irradiation field 42 of the X-ray image 41, and thus the table operation unit 51 can be used to perform operations more intuitively.

In this embodiment, as described above, the X-ray fluoroscopic imaging apparatus 100 further includes the collimator 5 (irradiation field adjuster) to adjust the X-ray irradiation field 42, and the main controller 31 is configured or programmed to, in the second control, perform a control to cause the collimator 5 to adjust the irradiation field 42 based on an operation input to the table operation unit 51 to move the center 42a of the irradiation field 42 of the X-ray image 41 on the display 40. Accordingly, the irradiation field 42 is adjusted to be narrowed, and thus areas that do not need to be visually recognized can be excluded from the imaging range. Therefore, the X-ray irradiation range can be reduced, and thus the amount of X-ray exposure to the subject can be reduced.

In this embodiment, as described above, the main controller 31 is configured or programmed to, in the second control, align the center 42a of the irradiation field 42 of the X-ray image 41 with the center 40a of the display 40 when moving the center 42a of the irradiation field 42 of the X-ray image 41 on the display 40. Accordingly, based on the moving direction indicated by the operation input to the table operation unit 51, the table 2, which is prohibited from moving, can be visually recognized on the display 40 as if it were moving in the same direction as the above-described moving direction. Therefore, operations can be performed more intuitively using the table operation unit 51, and the visibility of the region of interest in the X-ray image 41 can be improved.

In this embodiment, as described above, the collimator 5 includes the shielding vane to adjust the irradiation field 42 by moving in a predetermined direction, and the main controller 31 is configured or programmed to, in the second control, move the center 42a of the irradiation field 42 of the X-ray image 41 on the display 40 in the direction opposite to the moving direction indicated by the operation input to the table operation unit 51 by moving the shielding vane. By moving the shielding vane, the table 2, which is prohibited from moving, can be visually recognized on the display 40 as if it were moving, and thus operations can be performed more intuitively using the table operation unit 51.

In this embodiment, as described above, the main controller 31 is configured or programmed to, in the second control, move the center 42a of the irradiation field 42 of the X-ray image 41 on the display 40 in the direction opposite to the moving direction indicated by the operation input to the table operation unit 51, in accordance with the amount of movement of the shielding vane. Accordingly, the main controller 31 can appropriately move the center 42a of the irradiation field 42 of the X-ray image 41 on the display 40 in accordance with the amount of movement of the shielding vane by associating the amount of operation input to the table operation unit 51 with the amount of movement of the shielding vane.

In this embodiment, as described above, the shielding vane includes the first shielding vane 11 to narrow the irradiation field 42 from the first direction to the second direction opposite to the first direction, and the second shielding vane 12 movable independently of the first shielding vane 11 to narrow the irradiation field 42 from the second direction to the first direction, and the main controller 31 is configured or programmed to, in the second control, move the center 42a of the irradiation field 42 of the X-ray image 41 on the display 40 in the second direction by moving the first shielding vane 11 or the second shielding vane 12 in the second direction to adjust the irradiation field 42, based on the operation input in the first direction to the table operation unit 51, and move the center 42a of the irradiation field 42 of the X-ray image 41 on the display 40 in the first direction by moving the first shielding vane 11 or the second shielding vane 12 in the first direction to adjust the irradiation field 42, based on the operation input in the second direction to the table operation unit 51. By moving the first shielding vane 11 or the second shielding vane 12 to adjust the irradiation field 42, the moving direction indicated by the operation input to the table operation unit 51 can coincide with the moving direction of the irradiation field 42 of the X-ray image 41, and thus operations can be performed more intuitively using the table operation unit 51.

In this embodiment, as described above, the main controller 31 is configured or programmed to move only one of the first shielding vane 11 and the second shielding vane 12 in the second control. Accordingly, by moving only one of the first shielding vane 11 and the second shielding vane 12 and moving the center 42a of the irradiation field 42 of the X-ray image 41 on the display 40, the table 2, which is prohibited from moving, can be visually recognized on the display 40 as if it were moving. Therefore, operations can be performed more intuitively using the table operation unit 51.

### Modified Examples

The embodiment disclosed this time must be considered as illustrative in all points and not restrictive. The scope of the present invention is not shown by the above description of the embodiment but by the scope of claims for patent, and all modifications (modified examples) within the meaning and scope equivalent to the scope of claims for patent are further included.

For example, while the example in which in the second control, the main controller 31 performs a control to adjust the irradiation field 42 using the shielding vane based on the operation input to the table operation unit 51 to move the center 42a of the irradiation field 42 of the X-ray image 41 on the display 40 has been shown in the aforementioned embodiment, the present invention is not limited to this. In the present invention, the center 42a of the irradiation field 42 of the X-ray image 41 on the display 40 may be moved by sliding (shifting) the X-ray image 41 on the display 40 in the Y direction without performing a control to adjust the irradiation field 42 using the shielding vane.

While the example in which the imaging range of the X-ray image 41 in the initial state displayed on the display 40 is substantially the same as the detection range of the FPD of the X-ray detector 4 has been shown in the aforementioned embodiment, the present invention is not limited to this. In the present invention, the imaging range of the X-ray image 41 in the initial state displayed on the display 40 may be smaller than the detection range of the FPD of the X-ray detector 4, and is not particularly limited. For example, the imaging range of the X-ray image 41 in the initial state displayed on the display 40 may be 70% or 90% of the detection range of the FPD of the X-ray detector 4.

For example, an operation input to the table operation unit 51 and display of the X-ray image 41 in the second control are described in a case in which the imaging range of the X-ray image 41 in the initial state displayed on the display 40 is 70% of the detection range of the FPD of the X-ray detector 4. As an example of a modified example, display of the X-ray image 41 on the display 40 based on an operation input by tilting the table operation unit 51 in the first direction in the second control is described below.

When the imaging range of the X-ray image 41 in the initial state displayed on the display 40 is 70% of the detection range by the FPD of the X-ray detector 4, an X-ray shielded region corresponding to 15% of the first shielding vane 11 is formed at the right end of the display 40, and an X-ray shielded region corresponding to 15% of the second shielding vane 12 is formed at the left end of the display 40.

By tilting the table operation unit 51 in the first direction, an operation input in the first direction is executed on the table operation unit 51. The main controller 31 performs a control to move the first shielding vane 11 in the second direction and move the second shielding vane 12 in the second direction based on the operation input in the first direction to the table operation unit 51. That is, the main controller 31 performs a control to move the first shielding vane 11 and the second shielding vane 12 in the second direction until the X-ray shielded region corresponding to 15% of the second shielding vane 12 disappears at the left end of the display 40. Therefore, the X-ray shielded region corresponding to 15% at the left end of the display 40 becomes visible. Thereafter, the main controller 31 performs a control to move only the first shielding vane 11 in the second direction. The irradiation field 42 narrowed by the first shielding vane 11 and expanded by the second shielding vane 12 is displayed with a bias in the Y2 direction on the display 40.

Then, the main controller 31 performs a control to align the center 42a of the irradiation field 42 of the X-ray image 41 with the center 40a of the display 40 to display the irradiation field 42 displayed with a bias in the Y2 direction on the display 40 at the approximate center of the display 40 in the Y direction. Based on the operation input in the first direction to the table operation unit 51, the irradiation field 42 displayed with a bias in the Y2 direction on the display 40 is displayed at the approximate center of the display 40 in the Y direction such that the irradiation field 42 adjusted on the display 40 moves in the Y1 direction. Therefore, based on the operation input in the first direction to the table operation unit 51, the operator or technician can be caused to visually recognize on the display 40 the table 2, which is prohibited from moving, as if it were moving in the Y1 direction.

While the example in which the input 54 receives operation inputs to cause the table lock 7 to lock and unlock movement of the table 2 in the Y direction has been shown in the aforementioned embodiment, the present invention is not limited to this. In the present invention, the table lock 7 may be configured to prohibit movement of the table 2 in the Y direction based on an operation input to a treatment apparatus to start treatment or an operation input to an examination apparatus to start examination, and the main controller 31 may be configured or programmed to perform the second control based on an operation input to the table operation unit 51.

While the example in which the table operation unit 51 includes a joystick has been shown in the aforementioned embodiment, the present invention is not limited to this. In the present invention, the table operation unit 51 may include a component other than a joystick, such as a remote control lever or an operation panel including a plurality of operation buttons.

While the example in which the first shielding vane operation unit 52 and the second shielding vane operation unit 53 each include knobs that are movable in the forward-rearward direction has been shown in the aforementioned embodiment, the present invention is not limited to this. The first shielding vane operation unit 52 and the second shielding vane operation unit 53 each may include an operation panel including a plurality of operation buttons, for example.

While the example in which the collimator 5 includes the first to eighth shielding vanes 11 to 19 has been shown in the aforementioned embodiment, the present invention is not limited to this. The number of shielding vanes included in the collimator 5 and their independent/symmetric operation are not particularly limited.

While the example in which in the second control, the main controller 31 aligns the center 42a of the irradiation field 42 of the X-ray image 41 with the center 40a of the display 40 when moving the center 42a of the irradiation field 42 of the X-ray image 41 on the display 40 has been shown in the aforementioned embodiment, the present invention is not limited to this. In the present invention, when the center 42a of the irradiation field 42 of the X-ray image 41 on the display 40 is moved, the center 42a of the irradiation field 42 of the X-ray image 41 may not be aligned with the center 40a of the display 40.

While the example in which in the second control, the main controller 31 increases or reduces the size of the adjusted irradiation field 42 to match the size of the adjusted irradiation field 42 with the screen size of the display 40 when the center 42a of the irradiation field 42 of the X-ray image 41 on the display 40 has been moved has been shown in the aforementioned embodiment, the present invention is not limited to this. In the present invention, the size of the adjusted irradiation field 42 may not be increased or reduced to match the screen size of the display 40.

While the example in which in the second control, the main controller 31 moves the center 42a of the irradiation field 42 of the X-ray image 41 on the display 40 in accordance with the amount of movement of the shielding vane has been shown in the aforementioned embodiment, the present invention is not limited to this. In the present invention, the center 42a of the irradiation field 42 of the X-ray image 41 on the display 40 may be moved by a preset amount.

### ASPECTS

It will be appreciated by those skilled in the art that the exemplary embodiments described above are specific examples of the following aspects.

### Item 1

An X-ray fluoroscopic imaging apparatus comprising:
an imager including an X-ray irradiator to irradiate a subject with X-rays and an X-ray detector to detect the X-rays radiated from the X-ray irradiator;
an X-ray image generator to generate an X-ray image based on a detection signal detected by the X-ray detector;
a table between the X-ray irradiator and the X-ray detector to allow the subject to be placed thereon, the table being movable in a horizontal direction;
a table movement mechanism to move the table;
a table operation unit to receive an operation input to move the table;
a table lock to prohibit movement of the table;
a display to display the X-ray image generated by the X-ray image generator; and
a controller configured or programmed to control the movement of the table and perform a control to cause the display to display the X-ray image; wherein
the controller is configured or programmed to:
   move the table based on the operation input to the table operation unit in a first control performed when the movement of the table is not prohibited by the table lock; and
   move a center of an X-ray irradiation field of the X-ray image on the display without moving the table based on the operation input to the table operation unit in a second control performed when the movement of the table is prohibited by the table lock.

### Item 2

The X-ray fluoroscopic imaging apparatus according to item 1, wherein the controller is configured or programmed to move the center of the irradiation field of the X-ray image on the display in a direction opposite to a moving direction indicated by the operation input to the table operation unit in the second control.

### Item 3

The X-ray fluoroscopic imaging apparatus according to item 2, further comprising:
an irradiation field adjuster to adjust the X-ray irradiation field; wherein
the controller is configured or programmed to, in the second control, perform a control to cause the irradiation field adjuster to adjust the irradiation field based on the operation input to the table operation unit to move the center of the irradiation field of the X-ray image on the display.

### Item 4

The X-ray fluoroscopic imaging apparatus according to item 3, wherein the controller is configured or programmed to, in the second control, align the center of the irradiation field of the X-ray image with a center of the display when moving the center of the irradiation field of the X-ray image on the display.

### Item 5

The X-ray fluoroscopic imaging apparatus according to item 3, wherein
the irradiation field adjuster includes a shielding vane to adjust the irradiation field by moving in a predetermined direction; and
the controller is configured or programmed to, in the second control, move the center of the irradiation field of the X-ray image on the display in the direction opposite to the moving direction indicated by the operation input to the table operation unit by moving the shielding vane.

### Item 6

The X-ray fluoroscopic imaging apparatus according to item 5, wherein the controller is configured or programmed to, in the second control, move the center of the irradiation field of the X-ray image on the display in the direction opposite to the moving direction indicated by the operation input to the table operation unit, in accordance with an amount of movement of the shielding vane.

### Item 7

The X-ray fluoroscopic imaging apparatus according to item 5, wherein
the shielding vane includes a first shielding vane to narrow the irradiation field from a first direction to a second direction opposite to the first direction, and a second shielding vane movable independently of the first shielding vane to narrow the irradiation field from the second direction to the first direction; and
the controller is configured or programmed to, in the second control:
   move the center of the irradiation field of the X-ray image on the display in the second direction by moving the first shielding vane or the second shielding vane in the second direction to adjust the irradiation field, based on the operation input in the first direction to the table operation unit; and
   move the center of the irradiation field of the X-ray image on the display in the first direction by moving the first shielding vane or the second shielding vane in the first direction to adjust the irradiation field, based on the operation input in the second direction to the table operation unit.

### Item 8

The X-ray fluoroscopic imaging apparatus according to item 7, wherein the controller is configured or programmed to move only one of the first shielding vane and the second shielding vane in the second control.

### DESCRIPTION OF REFERENCE NUMERALS

1: X-ray fluoroscopic imaging apparatus body
2: table
3: X-ray irradiator
4: X-ray detector
5: collimator
7: table lock
8: imager
11: first shielding vane
12: second shielding vane
25: table movement mechanism
31: main controller
32: X-ray image generator
40: display
40a: center of the display
41: X-ray image
42: irradiation field
42a: center of the irradiation field
50: operation console
51: table operation unit
100: X-ray fluoroscopic imaging apparatus

1. An X-ray fluoroscopic imaging apparatus comprising:
an imager including an X-ray irradiator to irradiate a subject with X-rays and an X-ray detector to detect the X-rays radiated from the X-ray irradiator;
an X-ray image generator to generate an X-ray image based on a detection signal detected by the X-ray detector;
a table between the X-ray irradiator and the X-ray detector to allow the subject to be placed thereon, the table being movable in a horizontal direction;
a table movement mechanism to move the table;
a table operation unit to receive an operation input to move the table;
a table lock to prohibit movement of the table;
a display to display the X-ray image generated by the X-ray image generator; and
a controller configured or programmed to control the movement of the table and perform a control to cause the display to display the X-ray image; wherein
the controller is configured or programmed to:
move the table based on the operation input to the table operation unit in a first control performed when the movement of the table is not prohibited by the table lock,
move a center of an X-ray irradiation field of the X-ray image on the display without moving the table based on the operation input to the table operation unit in a second control performed when the movement of the table is prohibited by the table lock, and
move the center of the irradiation field of the X-ray image on the display in a direction opposite to a direction indicated by the operation input to the table operation unit in the second control.

2. (canceled)

3. The X-ray fluoroscopic imaging apparatus according to claim 1, further comprising:
an irradiation field adjuster to adjust the X-ray irradiation field; wherein
the controller is configured or programmed to, in the second control, perform a control to cause the irradiation field adjuster to adjust the irradiation field based on the operation input to the table operation unit to move the center of the irradiation field of the X-ray image on the display.

4. The X-ray fluoroscopic imaging apparatus according to claim 3, wherein the controller is configured or programmed to, in the second control, align the center of the irradiation field of the X-ray image with a center of the display when moving the center of the irradiation field of the X-ray image on the display.

5. The X-ray fluoroscopic imaging apparatus according to claim 3, wherein the irradiation field adjuster includes a shielding vane to adjust the irradiation field by moving in a predetermined direction; and
the controller is configured or programmed to, in the second control, move the center of the irradiation field of the X-ray image on the display in the direction opposite to the moving direction indicated by the operation input to the table operation unit by moving the shielding vane.

6. The X-ray fluoroscopic imaging apparatus according to claim 5, wherein the controller is configured or programmed to, in the second control, move the center of the irradiation field of the X-ray image on the display in the direction opposite to the direction indicated by the operation input to the table operation unit, in accordance with an amount of movement of the shielding vane.

7. The X-ray fluoroscopic imaging apparatus according to claim 5, wherein
the shielding vane includes a first shielding vane to narrow the irradiation field from a first direction to a second direction opposite to the first direction, and a second shielding vane movable independently of the first shielding vane to narrow the irradiation field from the second direction to the first direction; and
the controller is configured or programmed to, in the second control:
move the center of the irradiation field of the X-ray image on the display in the second direction by moving the first shielding vane or the second shielding vane in the second direction to adjust the irradiation field, based on the operation input in the first direction to the table operation unit; and
move the center of the irradiation field of the X-ray image on the display in the first direction by moving the first shielding vane or the second shielding vane in the first direction to adjust the irradiation field, based on the operation input in the second direction to the table operation unit.

8. The X-ray fluoroscopic imaging apparatus according to claim 7, wherein the controller is configured or programmed to move only one of the first shielding vane and the second shielding vane in the second control.
